(19) United States
(12) Patent Application Publication
(10) Pub. No.: US 2025/0332328 A1
(43) Pub. Date: Oct. 30, 2025

(54) Sampling Methods And Devices For Medical Equipment

(71) Applicant: Terumo BCT, Inc., Lakewood, CO (US)

(72) Inventors: Thomas J. FELT, Boulder, CO (US); John PITTINGER, Evergreen, CO (US); Dennis J. HLAVINKA, Lakewood, CO (US)

(21) Appl. No.: 19/189,852

(22) Filed: Apr. 25, 2025

Related U.S. Application Data
(60) Provisional application No. 63/639,853, filed on Apr. 29, 2024.

(51) Int. Cl. A61M 1/02

(52) U.S. Cl. CPC A61M 1/0236; A61M 2205/12; A61M 2205/18; A61M 2205/3393; A61M 2205/6072

(57) ABSTRACT

A method for in-line sampling for a medical device includes prompting an operator of a medical device to establish a fluid communication between a fluid component collection set as received by the medical device and a sample system for sample collection, where the medical device prohibits removal of the fluid component collection set or a component thereof from fluid communication with the medical device until sample collection using the sample system is complete and the establishing of fluid communication between the fluid component collection set includes prompting the medical device to interpret a code disposed on an exterior-facing surface of the sample tube.

### CROSS-REFERENCE TO RELATED APPLICATIONS

This application claims the benefit of U.S. Provisional Application No. 63/639,853 filed on Apr. 29, 2024. The entire disclosure of the above application is incorporated herein by reference.

### FIELD

The present disclosure relates to sampling methods and devices for medical equipment, like apheresis devices and systems.

### BACKGROUND

This section provides background information related to the present disclosure which is not necessarily prior art.

There are two common methods for blood donation and collection. The first method includes obtaining whole blood donation from a donor or subject. Once the whole blood is obtained a centrifugal process is often used to separate blood components or constituents from the whole blood. The components or constituents may be separated based on the different densities of the blood components or constituents. The desired components can be manually, semi-automatically, or automatically moved to a collection container during and/or after application of the centrifugal forces.

The second method is often referred to as an apheresis collection and requires specialized machine. A pheresis collection includes extracting whole blood from a donor or subject while connected to the specialized machine. The specialized machine separates the whole blood into various blood components or constituents to collect only the desired or targeted blood component(s) or constituent(s) and returns all other blood component(s) or constituent(s) to the donor or subject during the same donation connection or cycle. While the donor is connected to the apheresis machine, and during collection, it is often desirable to sample the collected blood component(s) or constituent(s) to ensure sample consistency and quality, to ensure and enforce safe and accepted collection levels, and to monitor collection processes. The position of a sample or product or collection container during the apheresis collection often prevents in situ sampling. It would be desirable to development inline sampling methods and devices for apheresis collection systems.

### SUMMARY

This section provides a general summary of the disclosure and is not a comprehensive disclosure of its full scope or all of its features.

At least one example embodiment relates to an in-line sampling method for a medical device.

In at least one example embodiment, the in-line sampling method includes establishing fluid communication between a fluid component collection set as received by the medical device and a sample system for sample collection, where the medical device prohibits removal of the fluid component collection set or a component thereof from fluid communication with the medical device until sample collection using the sample system is complete.

In at least one example embodiment, the sample collection includes transferring a sample volume to the sample system and the sample collection is complete only after entire sample volume is transferred.

In at least one example embodiment, the method may further include determining the sample volume based on a specific nature of a collection process using the medical device.

In at least one example embodiment, the medical device may include a scale and the sample collection is complete when the sample system is found to have a selected weight.

In at least one example embodiment, the method may further include prompting an operator to establish the fluid communication between the fluid component collection set and the sample system.

In at least one example embodiment, the method may further include alerting the operator when sample collection is complete.

In at least one example embodiment, the method may further include, concurrent with or after the alerting of the operator that the sample collection is complete, prompting the operator to remove the sample system from fluid communication with the medical device.

In at least one example embodiment, the prompt to the operator to removing the sample system from fluid communication with the medical device may include prompting the operator to remove the sample system from fluid communication with the fluid component collection set.

In at least one example embodiment, the method may further include, concurrent with or after the prompting of the operator to remove the sample system from fluid communication with the medical device, prompting the operator to remove a collection container from the medical device and from fluid communication with the fluid component collection set.

In at least one example embodiment, the medical device may prohibit removal of the collection container from the medical device and from fluid communication with the fluid component collection set until the sample collection using the sample system is complete.

In at least one example embodiment, the method may further include, after the removal of the collection container from the medical device, prompting the operator to remove the fluid component collection set from fluid communication with the medical device.

In at least one example embodiment, the method may further include, before the establish of the fluid communication between the fluid component collection set as received by the medical device and the sample system for sample collection, prompting an operator to confirm removal of one or more connectors from a subject, the one or more connectors fluidly connecting the medical device and the subject for the sample collection.

In at least one example embodiment, the sampling system may include a sample sleeve having a first end and an opposing second end, where the first end is couplable to the fluid component collection set.

In at least one example embodiment, the sampling system may further include a sample tube configured to be at least partially received by the second end of the sample sleeve.

In at least one example embodiment, the sampling system may further include a sealing member for securing the sample tube within the sample sleeve while allowing for movement of the sample tube from a connected state where there is fluid communication between the sample tube and the fluid component collection set and a disconnected state where there is no fluid communication between the sample tube and the fluid component collection set.

In at least one example embodiment, the first end of the sample sleeve may be couplable to the fluid component collection set using a tee connector.

In at least one example embodiment, the tee connector may be provided with the fluid component collection set.

In at least one example embodiment, the first end of the sample sleeve may include a cap and a conduit that extends therethrough. The conduit may include a first end and an opposing second end, where the first end of the conduit is configured to couple the sample sleeve to the fluid component collection set and the second end of the conduit is configured to engage with and extend into the sample tube.

In at least one example embodiment, the method may further include prompting the medical device to interpret a code disposed on an exterior-facing surface of the sample tube.

At least one example embodiment relates to another in-line sampling method for a medical device.

In at least one example embodiment, the method may include prompting an operator of a medical device to establish a fluid communication between a fluid component collection set as received by the medical device and a sample tube for sample collection, where the medical device prohibits removal of the fluid component collection set or a component thereof from fluid communication with the medical device until sample collection using the sample tube is complete and the establishing of fluid communication between the fluid component collection set includes prompting the medical device to interpret a code disposed on an exterior-facing surface of the sample tube.

In at least one example embodiment, the fluid component collection set may include a sample sleeve configured to receive the sample tube and a sealing member for securing the sample tube within the sample sleeve while allowing for movement of the sample tube from a connected state where there is fluid communication between the sample tube and the fluid component collection set and a disconnected state where there is no fluid communication between the sample tube and the fluid component collection set.

In at least one example embodiment, the method may further include, before the prompting of the operator to establish fluid communication between the fluid component collection set as received by the medical device and the sample tube for sample collection, prompting the operator to confirm removal of one or more connectors from a subject, the one or more connectors fluidly connecting the medical device and the subject for the sample collection.

In at least one example embodiment, the method may further include alerting the operator when sample collection is complete and prompting the operator to remove the sample tube from fluid communication with the medical device.

In at least one example embodiment, the medical device may include a scale and the sample collection is complete when the sample tube is found to have a selected weight.

At least one example embodiment relates to another in-line sampling method for a medical device.

In at least one example embodiment, the method may include prompting an operator of the medical device to establish fluid communication between a fluid component collection set received by the medical device and a sample tube for sample collection.

In at least one example embodiment, the method may further include, after the sample collection, prompting the operator to remove the sample tube from fluid communication with the fluid component collection set.

In at least one example embodiment, the method may further include, after the sample collection, prompting the operator to remove a collection container from the medical device.

In at least one example embodiment, before the prompting of the operator to establish fluid communication, the method may further include, prompting the operator to confirm removal of one or more connectors from a subject, where the one or more connectors fluidly connect the medical device and the subject for a collection process using the medical device.

At least one example embodiment relates to another in-line sampling method for a medical device.

In at least one example embodiment, the method may include identifying a sampling system. The sampling system may include a sample sleeve having a first end and an opposing second end, where the first end is couplable to a fluid component collection set. The sampling system may further include a sample tube configured to be at least partially received by the second end of the sample sleeve and a sealing member for securing the sample tube within the sample sleeve while allowing for movement of the sample tube from a connected state where there is fluid communication between the sample tube and the fluid component collection set and a disconnected state where there is no fluid communication between the sample tube and the fluid component collection set. The method may further include prompting an operator of the medical device to establish fluid communication between the fluid component collection set as received by the medical device and the sampling system.

In at least one example embodiment, the identifying of the sampling system may include reading a code disposed on exterior-facing surface of the sample tube.

At least one example embodiment relates to a sampling system of sampling a medical device.

In at least one example embodiment, the sampling system may include a sample sleeve having a first end and an opposing second end, where the first end is couplable to a fluid component collection set. The sampling system may further include a sample tube configured to be at least partially received by the second end of the sample sleeve and a sealing member for securing the sample tube within the sample sleeve while allowing for movement of the sample tube from a connected state where there is fluid communication between the sample tube and the fluid component collection set and a disconnected state where there is no fluid communication between the sample tube and the fluid component collection set.

In at least one example embodiment, the first end of the sample sleeve may be couplable to the fluid component collection set using a tee connector.

In at least one example embodiment, the first end of the sample sleeve may include a cap and a conduit that extends therethrough. The conduit may include a first end and an opposing second end. The first end of the conduit may be configured to couple the sample sleeve to the fluid component collection set. The second end of the conduit may be configured to engage with and extend into the sample tube.

In at least one example embodiment, the sampling system may further include a code disposed on an exterior-facing surface of the sample tube. The code may be configured to identify the sampling system.

Further areas of applicability will become apparent from the description provided herein. The description and specific examples in this summary are intended for purposes of illustration only and are not intended to limit the scope of the present disclosure.

DRAWINGS

The drawings described herein are for illustrative purposes only of selected embodiments and not all possible implementations and are not intended to limit the scope of the present disclosure.

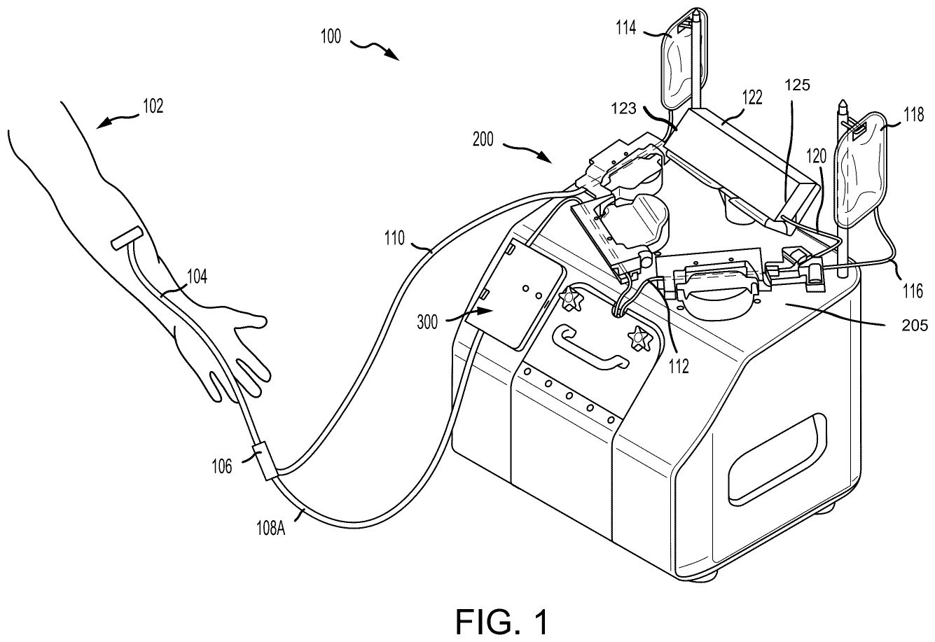

FIG. 1 is a perspective view of an operating environment of an example apheresis system in accordance with at least one example embodiment;

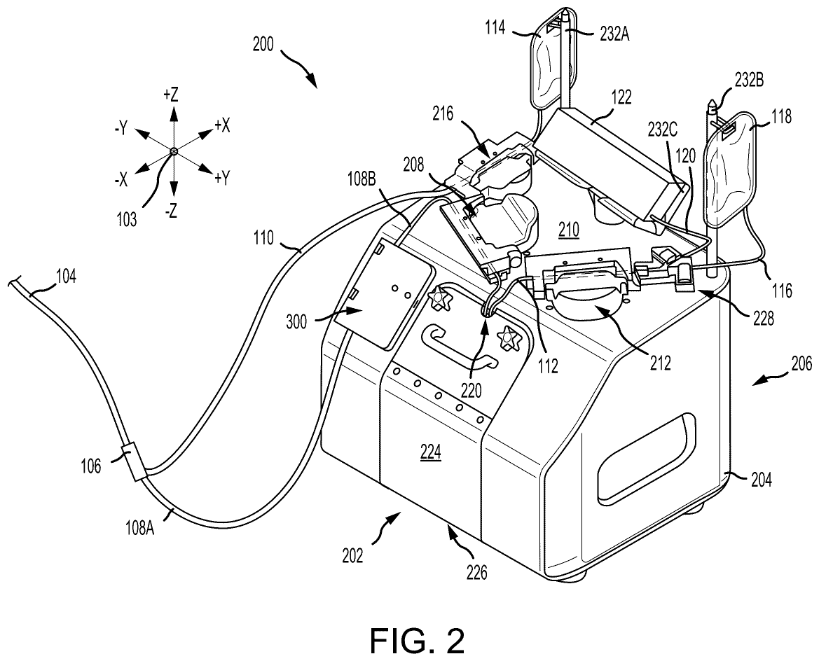

FIG. 2 is a perspective view of the apheresis system illustrated in FIG. 1, where the access door is in a closed position in accordance with at least one example embodiment;

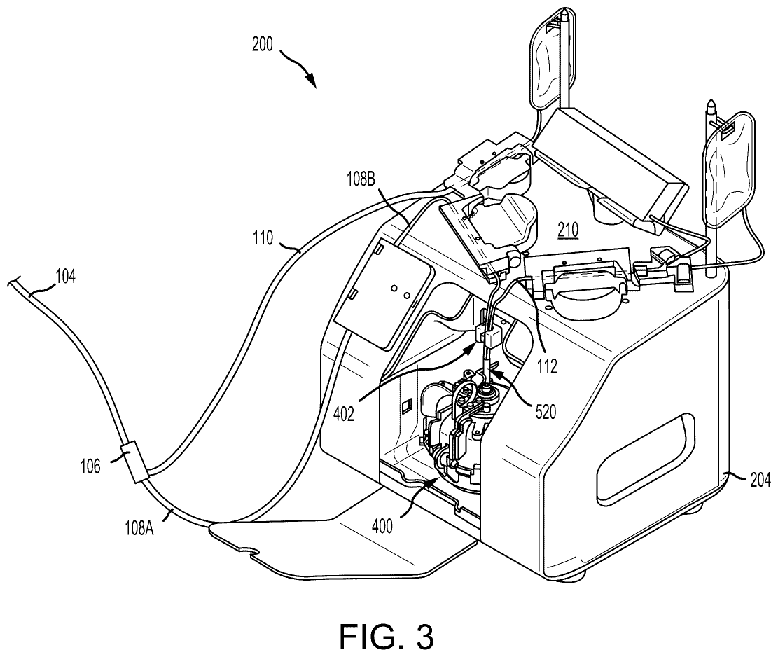

FIG. 3 is a perspective view the apheresis system illustrated in FIG. 1, where the access door is in an open position revealing a centrifuge assembly in accordance with at least one example embodiment;

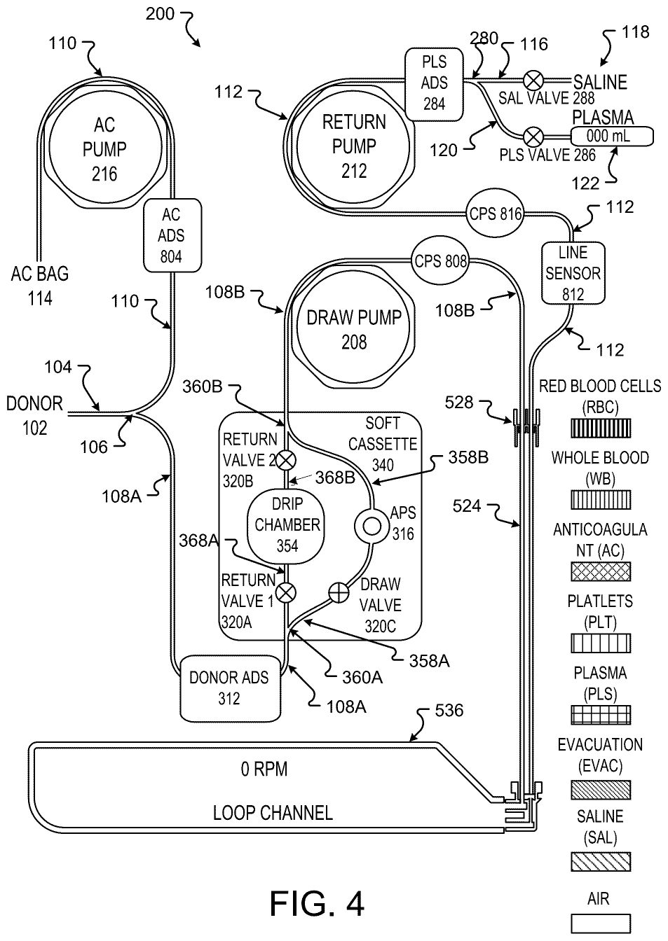

FIG. 4 is a functional diagram of for the apheresis system illustrated in FIG. 1 in accordance with at least one example embodiment;

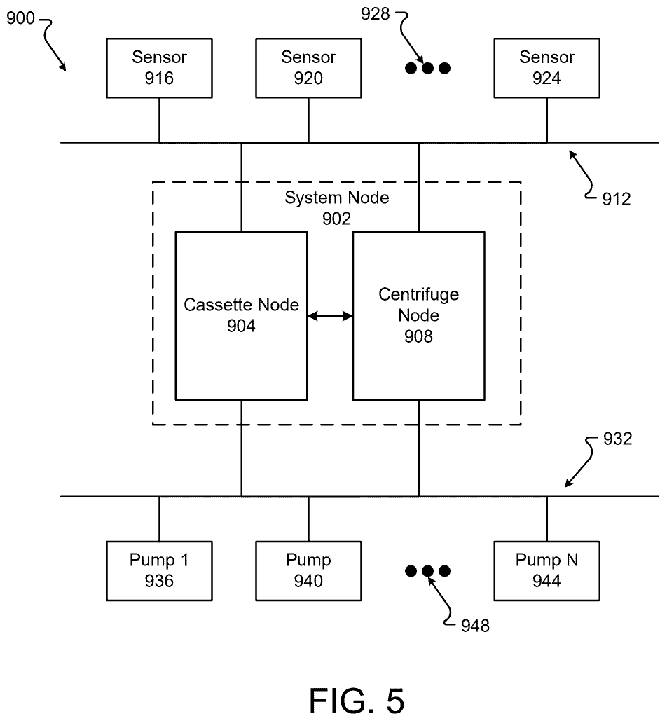

FIG. 5 is a block diagram of an electrical system of the apheresis system illustrated in FIG. 1 in accordance with at least one example embodiment;

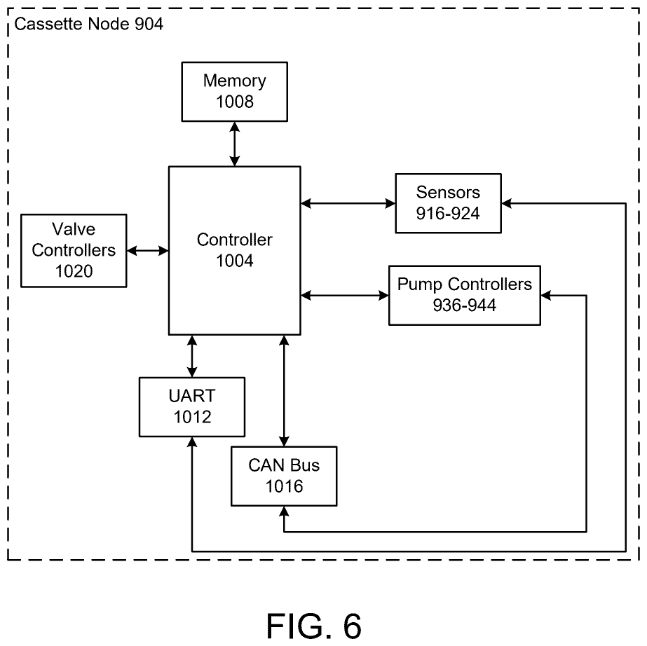

FIG. 6 is a further block diagram of the electrical system of FIG. 5 in accordance with at least one example embodiment;

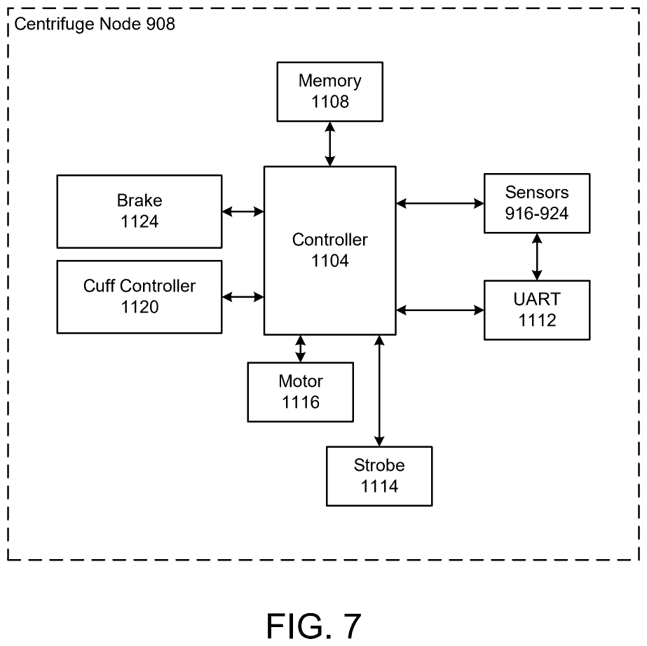

FIG. 7 is a further block diagram of the electrical system of FIG. 5 in accordance with at least one example embodiment;

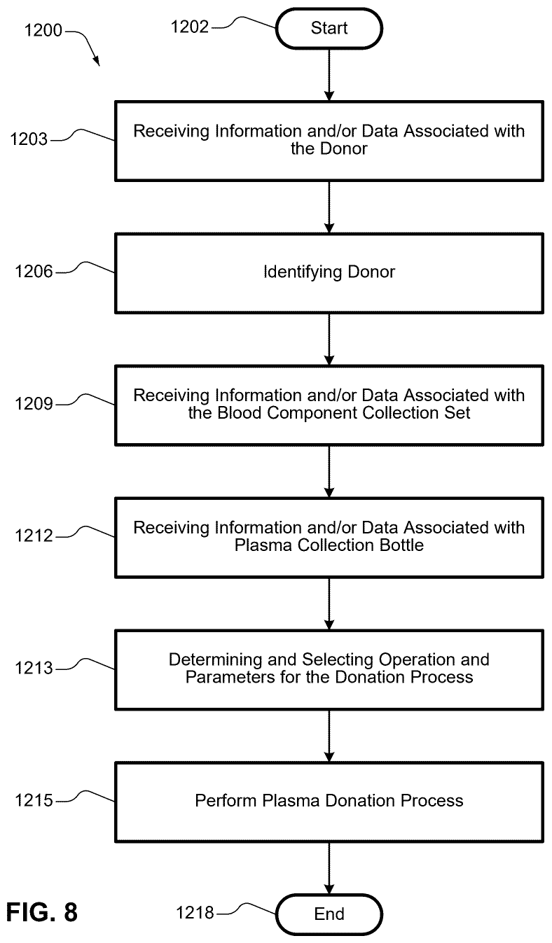

FIG. 8 is a flowchart illustrating an example method of using the apheresis system illustrated in FIG. 1 in accordance with at least one example embodiment;

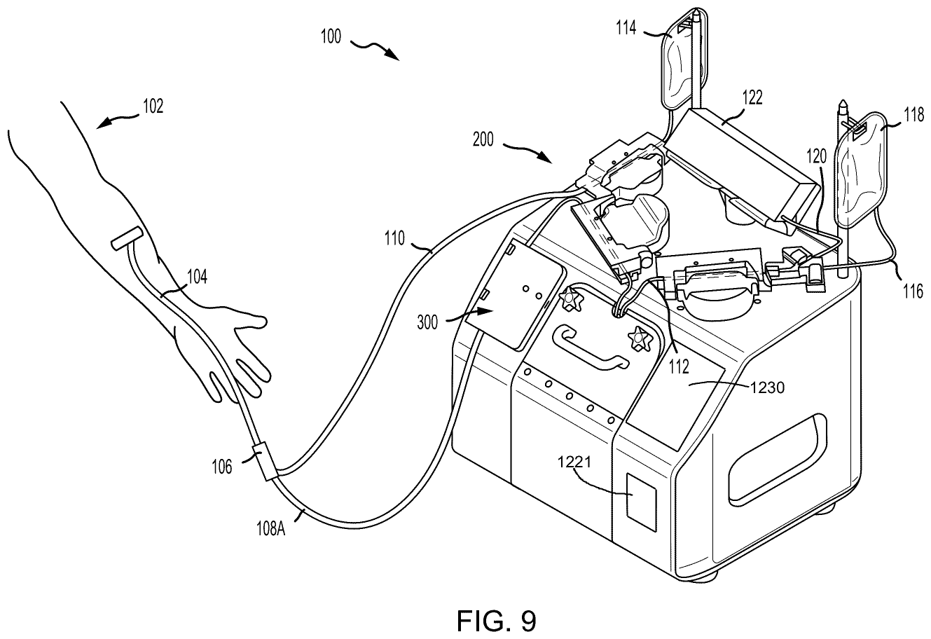

FIG. 9 is a perspective view of the apheresis system illustrated in FIG. 1 further including a scanner in accordance with at least one example embodiment;

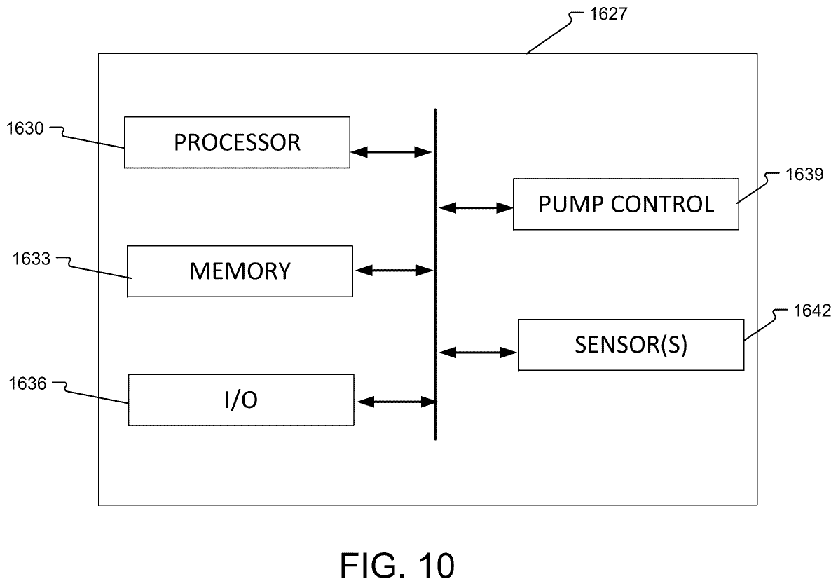

FIG. 10 is a block diagram of an example computing system of the apheresis system illustrated in FIG. 1 in accordance with at least one example embodiment;

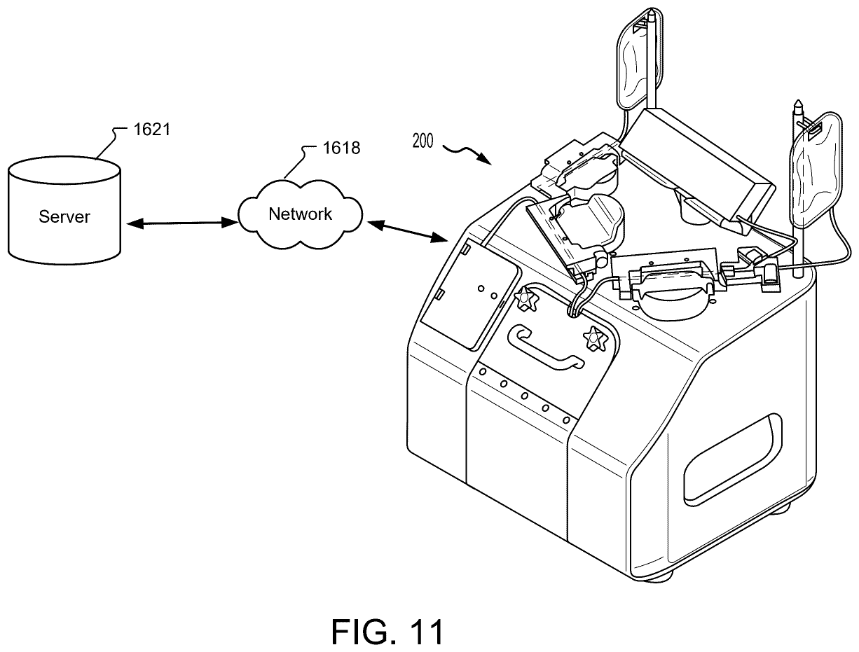

FIG. 11 shows the apheresis system illustrated in FIG. 1 in communication with a network in accordance with at least one example embodiment;

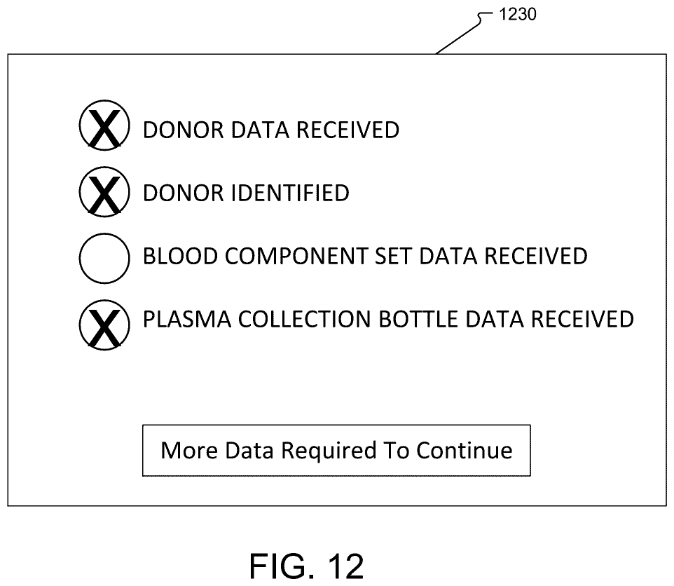

FIG. 12 illustrates an example graphical user interface for the apheresis system illustrated in FIG. 1 in accordance with at least one example embodiment;

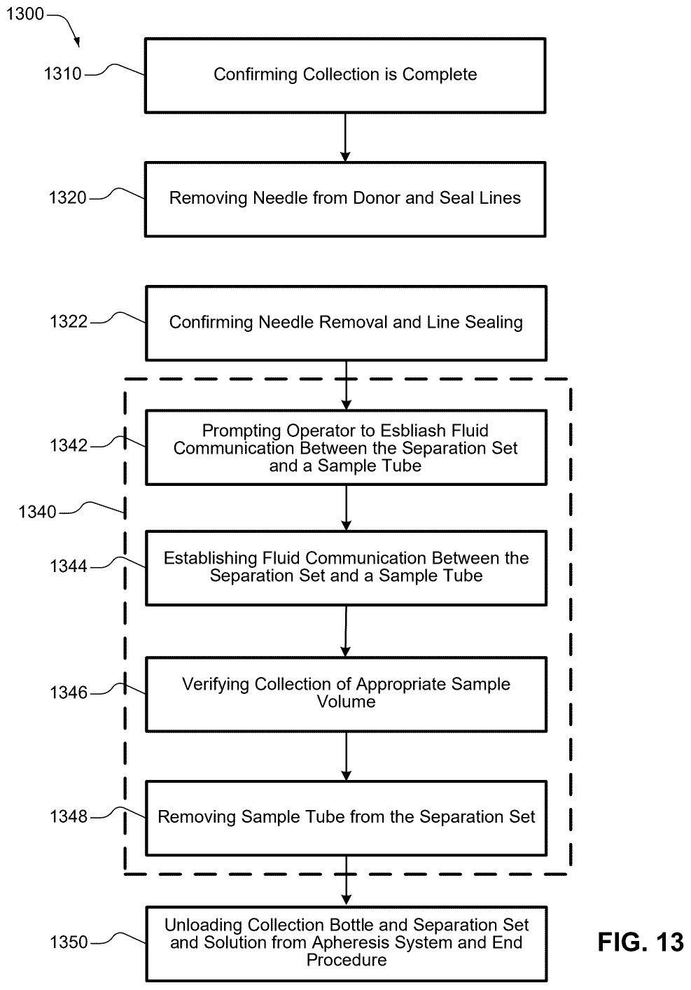

FIG. 13 is a flowchart illustrating an example inline sampling method for use with apheresis systems, like the apheresis system illustrated in FIG. 1, in accordance with at least one example embodiment;

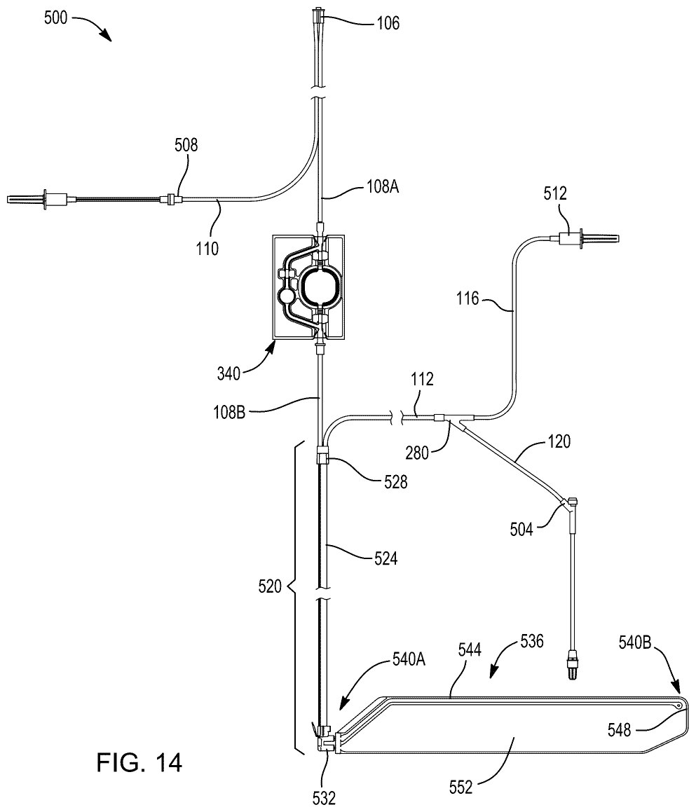

FIG. 14 is an illustration of an example fluid component collection set that can be used with an apheresis system, like the apheresis system illustrated in FIG. 1, and for inline sampling methods, like the method illustrated in FIG. 13, in accordance with at least one example embodiment; and

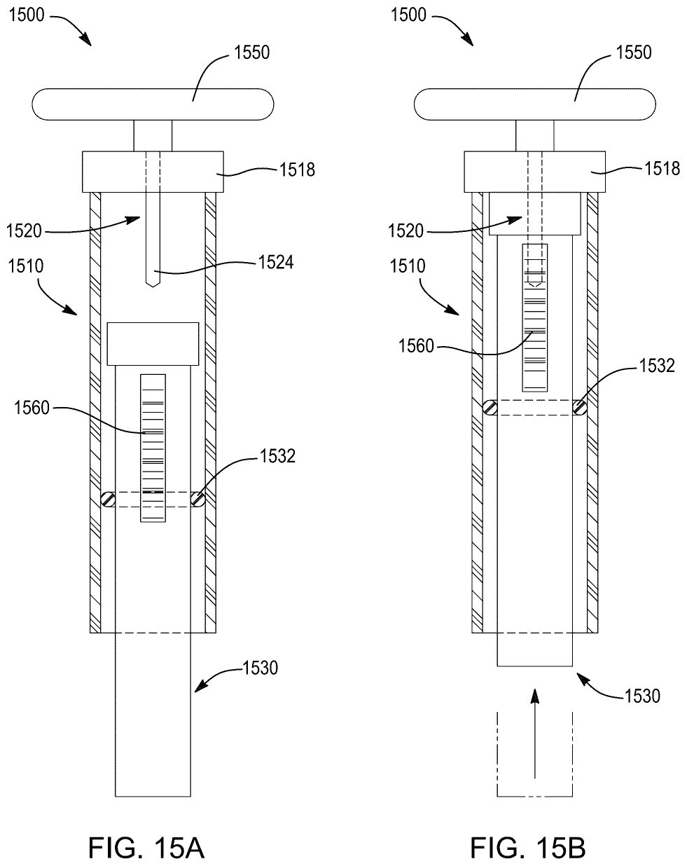

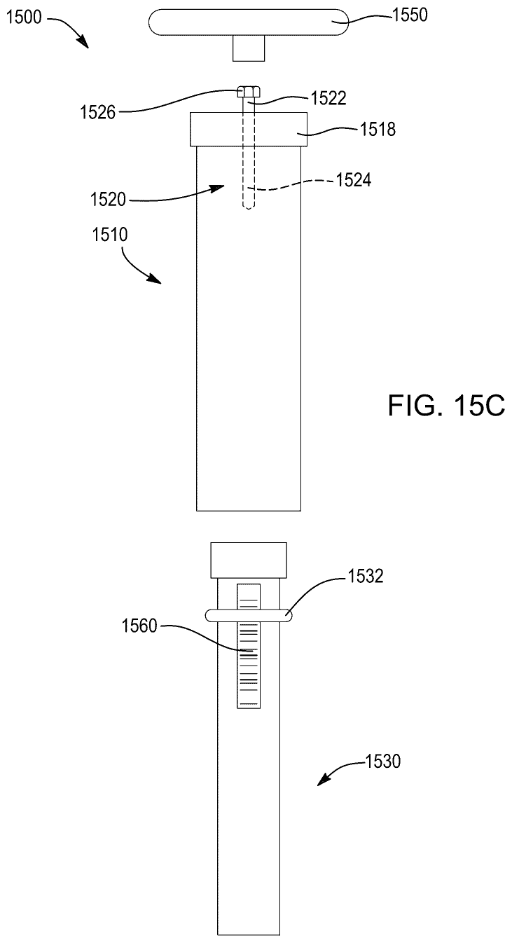

FIGS. 15A-15C are illustrations of an example sampling system that can be used for inline sampling methods, like the method illustrated in FIG. 13, in accordance with at least one example embodiment.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

In this application, including the definitions below, the term “module” or the term “controller” may be replaced with the term “circuit.” The term “module” may refer to, be part of, or include: an Application Specific Integrated Circuit (A SIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

The present disclosure relates to methods of and means for collecting one or more blood components or constituents, like plasma, using medical equipment. The medical equipment may include apheresis devices or systems like those detailed in U.S. Pat. No. 11,090,425, titled METHODS AND SYSTEMS FOR HIGH-THROUGHOUT BLOOD COMPONENT COLLECTION and issued Aug. 17, 2021; U.S. application Ser. No. 18/117,035, titled SOFT CASSETTE WITH INTEGRATED FEATURES and filed Mar. 3, 2023; U.S. application Ser. No. 18/116,919, titled BLOOD COMPONENT COLLECTION BLADDER and filed Mar. 3, 2023; U.S. application Ser. No. 18/117,077, titled INTEGRATED CODE SCANNING SYSTEM AND APHERESIS DATA CONTROL METHOD and filed Mar. 3, 2023; U.S. application Ser. No. 18/117,044, titled METHODS AND SYSTEMS FOR THE CALIBRATION, MAINTENANCE, AND SERVICE OF APHERESIS SYSTEMS and filed Mar. 3, 2023; U.S. application Ser. No. 18/116,902, titled MOVING BLOOD COMPONENT COLLECTION LOOP HOLDER and filed Mar. 3, 2023; U.S. application Ser. No. 18/116,958, titled BOTTLE TRAY WITH MAGNETIC COUPLING AND LOAD CELL OVERLOAD PROTECTION and filed Mar. 3, 2023; U.S. application Ser. No. 18/116,988, titled COMMUNICATIONS AND OPERATION CONTROL OF APHERESIS SYSTEMS and filed Mar. 3, 2023; U.S. application Ser. No. 18/117,006, titled METHODS AND INTERFACES FOR PROVIDING DONATION PROCESS FEEDBACK and filed Mar. 3, 2023; U.S. application Ser. No. 18,117,007, titled MODULAR SERVICEABILITY SLEDS AND INTERCONNECTIONS and filed Mar. 3, 2023; U.S. application Ser. No. 18/116,908, titled COLLECTION BOTTLE WITH INTEGRATED CAP, HANDLE, AND SHIELD FEATURES and filed Mar. 3, 2023; U.S. application Ser. No. 18/116,992, titled METHODS FOR PROVIDING AUTOMATIC FLOW ADJUSTMENTS and filed Mar. 3, 2023; U.S. application Ser. No. 18/116,999, titled APHERESIS SYSTEM SAFETY FEATURES and filed Mar. 3, 2023; U.S. application Ser. No. 18/117,029, titled AUTOMATIC OPERATIONAL CONTROL BASED ON DETECTED ENVIRONMENTAL STATE and filed Mar. 3, 2023; U.S. application Ser. No. 18/116,954, titled FLEXURE-BASED TUBING STATE SENSOR and filed Mar. 3, 2023; U.S. application Ser. No. 18/117,035, titled SOFT CASSETTE WITH INTEGRATED FEATURES and filed Mar. 3, 2023; and U.S. application Ser. No. 18/117,073, titled BLOOD COMPONENT COLLECTION SET WITH INTEGRATED SAFETY FEATURES and filed Mar. 3, 2023, the entire disclosures of which are hereby incorporated by references.

A pheresis systems generally include one or more connections configured to move whole blood and/or blood components or constituents to and from a blood component separation device housed within the apheresis system, where the blood component separation device is a centrifuge. FIG. 1 is a perspective view of an operating environment 100 of an example apheresis system 200. The operating environment 100 may include an apheresis system 200, a donor or subject 102, and one or more connections (eg., feed tubing 104, cassette inlet tubing 108A, anticoagulant (AC) tubing 110, etc.) running from the donor or subject 102 to the apheresis system 200 and/or vice versa. The feed tubing 104 may be fluidly connected with at least one blood vessel, for example, a vein, of the donor or subject 102 via venipuncture. For example, a cannula connected to an end of the feed tubing 104 may be inserted through the skin of the donor or subject 102 and into a target site, or vein. This connection may provide an intravenous path for blood to flow from the donor or subject 102 to the apheresis system 200 and/or for blood components or constituents to flow back to the donor or subject 102. The whole blood supplied from the donor or subject 102 may flow along the feed tubing 104 through a tubing connector 106 and along the cassette inlet tubing 108A into a soft cassette assembly 300. The soft cassette assembly 300 may include one or more fluid control paths and valves for selectively controlling the flow of blood to and/or from the donor or subject 102. The apheresis system 200 may include an anticoagulant supply contained in an anticoagulant bag 114. The anticoagulant may be pumped at least through the anticoagulant tubing 110 and the tubing connector 106 preventing the coagulation of blood in the apheresis system 200.

FIG. 2 is a perspective view of the apheresis system 200 described in FIG. 1. The apheresis system 200 may provide for a continuous whole blood separation process. In at least one example embodiment, the whole blood may be withdrawn from the donor or subject 102 and substantially continuously provided to a blood component separation device of the apheresis system 200 where the blood may be separated into various components or constituents and at least one of these blood components or constituents may be collected from the apheresis system 200. In at least one example embodiment, one or more of the separated blood components or constituents may be either collected, for subsequent use, or returned to the donor or subject 102. The whole blood may be withdrawn from the donor or subject 102 and directed into a centrifuge of the apheresis system 200 through an opening 220 in an access panel 224 of the apheresis system 200. In at least one example embodiment, the tubing the feed tubing 104, the cassette inlet tubing 108A, inlet tubing 108B (also referred to herein as loop inlet tubing 108B), exit tubing 112 (also referred to herein as loop exit tubing 112), the saline tubing 116, and the plasma tubing 120, used in the extracorporeal tubing circuit may together define a closed, sterile, and disposable system (which may also be referred to as an exchange set and/or a blood component collection set and/or a fluid component collection set).

Operation of the various pumps, valves, and blood component separation device (eg., centrifuge), may be controlled by one or more processors included in the apheresis system 200, and may include a plurality of embedded computer processors that are part of a computer system. The computer system may also include components that allow a user to interface with the computer system, including for example, memory and storage devices (RAM, ROM (eg., CD-ROM, DVD), magnetic drives, optical drives, flash memory, etc.); communication/networking devices (eg., wired such as modems/network cards, or wireless such as Wi-Fi); input devices such as keyboard(s), touch screen(s), camera(s), and/or microphone(s); and output device(s) such as display(s), and audio system(s), etc. It should be appreciated that, in at least one example embodiment, for example, as illustrated in FIG. 9, to assist the operator of the apheresis system 200 with various aspects of its operation, the blood component separation device (e.g., centrifuge), may include a graphical user interface with a display that includes an interactive touch screen.

With renewed reference to FIG. 2, the apheresis system 200 may include a housing 204 and/or structural frame, a cover 210, an access panel 224 disposed at a front 202 and/or rear 206 of the apheresis system 200, and one or more supports 232A-232C including hooks, rests, cradles, arms, protrusions, plates, and/or other support features for holding, cradling, and/or otherwise supporting a container or the anticoagulant bag 114, the saline bag 118, or the collection container 122. The housing 204 may include a machine frame (e.g., made of welded, bolted, and/or connected structural elements; extruded material; and/or beams) to which one or more panels, such as the cover 210, doors, subassemblies, and/or components are attached. In at least one example embodiment, at least one panel of the apheresis system 200 may include a mounting surface for the soft cassette assembly 300, one or more pumps such as a draw pump 208, a return pump 212, an anticoagulant pump 216, and/or a fluid valve control system 228 (eg., plasma and saline valve control).

Further, the housing 204 and/or structural frame may be configured such that the collection container 122 as received has a general downward tilt relative to the planar top surface 250 of the housing 204. As illustrated, a first end 123 of the collection container 122 may have a first position and the second end 125 of the collection container 122 may have a second position, where a first distance between the first end 123 of the collection container 122 and the planar top surface 250 of the housing 204 is greater than a second distance between the season end 125 of the collection container 122 and planar top surface 250 of the housing 204. The housing 204 and/or structural frame may be configured such that the collection container 122 as received has a general downward tilt relative to the planar top surface of the housing 204 of greater than or equal to about 10 degrees to less than or equal to about 20 degrees.

The access panel 224 may include one or more handles, locks, and a pivoting or hinged axis 226 (eg., a door hinge, piano hinge, continuous hinge, cleanroom hinge, etc.). The access panel 224 may be selectively opened to provide access to an interior of the apheresis system 200, and more specifically, to a blood separation assembly, (eg., centrifuge assembly). For example, the access panel 224 may provide access to load and/or unload the centrifuge with one or more components in the fluid component collection set (eg., the collection set 500 illustrated in FIG. 14). In at least one example embodiment, the inside of the apheresis system 200 may be separated into at least a centrifuge portion and a controls portion. For example, the centrifuge portion may include a cavity configured to receive the centrifuge, rotation motor, and associated hardware. This area may be physically separated from the controls portion via one or more walls of the cavity. In at least one example embodiment, access to the controls portion (eg., configured to house or otherwise contain the motor controller, CPU or processor(s), electronics, and/or wiring) may be provided via a securely fastened panel of the housing 204 and/or panel separate from the access panel 224.

In at least one example embodiment, the apheresis system 200 may include a number of pumps, such as the draw pump 208, the return pump 212, and/or the anticoagulant pump 216, which can be configured to control the flow of fluid (e.g., blood and/or blood components, anticoagulant, and/or saline) through the apheresis system 200. As shown in FIG. 2, in at least one example embodiment, the draw pump 208, the return pump 212, and/or the AC pump 216 may be disposed at least partially on a top portion of the cover 210 of the apheresis system 200. In at least one example embodiment, the draw pump 208 may control blood flow to and/or from the donor or subject 102 into the centrifuge of the apheresis system 200. For example, the draw pump 208 may engage with a portion of the inlet tubing 108B disposed between the soft cassette assembly 300 and the centrifuge of the apheresis system 200. In at least one example embodiment, the return pump 212 may be configured to control a flow of separated blood components or constituents (eg., plasma) from the centrifuge to a collection container 122 and/or vice versa. Additionally, or alternatively, the return pump 212 may control a flow of saline (eg., supplied from the saline bag 118) throughout the fluid component collection set and/or apheresis system 200. In at least one example embodiment, the anticoagulant pump 216 may engage with a portion of the anticoagulant tubing 110 to selectively control the flow of anticoagulant throughout the fluid component collection set of the apheresis system 200.

FIG. 3 illustrates an example centrifuge assembly 400 for use with or in the apheresis system 200. For example, the centrifuge assembly 400 may be disposed in an interior space of the apheresis system 200. The interior space may be at least partially enclosed with one or more elements of the housing 204 and/or centrifuge chamber. Access to the interior space and the centrifuge assembly 400 may be provided via the access panel 224 disposed at the front 202 of the apheresis system 200. For example, in FIG. 2, the access panel 224 is shown in an open position, opened along the hinged axis 226. The hinged axis 226 may correspond to a door hinge, continuous hinge, cleanroom hinge, and/or other panel hinges.

With renewed reference to FIG. 3, the centrifuge assembly 400 may be operatively mounted inside the apheresis system 200 such that the assembly 400 is capable of rotating relative to the housing 204 and/or other elements of the apheresis system 200. The centrifuge assembly 400 may be loaded with one or more portions of the fluid component collection set by routing tubing (eg., the inlet tubing 108B and the exit tubing 112) into the interior space of the apheresis system 200 (e.g., via the opening 220 shown in FIG. 2), connecting a portion of a blood component collection loop 520 to the fixed loop connection 402 and inserting a blood component collection bladder into a filler 460. The fixed loop connection 402 maintains the inlet tubing 108B and the exit tubing 112 in a fixed position and may prevent twisting of the tubing 108B, 112 outside of the apheresis system 200. In at least one example embodiment, the blood component collection loop may be interconnected to the fixed loop connection 402 via one or more keyed features or positive location features.

FIG. 4 provides a functional diagram of the apheresis system 200. FIG. 4 shows the components as previously introduced in a functional diagram to describe the operation of the system 200 for extracting plasma or other blood components or constituents from the whole blood of the donor or subject 102 during an apheresis procedure or process.

As illustrated, the anticoagulant pump 216 may be configured to pump fluid in the anticoagulant tubing 110 from the anticoagulant bag 114. In at least one example embodiment, the anticoagulant tubing 110 may also include an anticoagulant air detection sensor (ADS) 804 configured to detect air or fluid within the anticoagulant tubing 110. The anticoagulant tubing 110 may intersect with and be fluidly associated with the feed tubing 104 and also the cassette inlet tubing 108A at tubing connector 106. The tubing connector 106 may be any type of connection configured to connect the different tube 110, 104, and/or 108A.

The feed tubing 104 proceeds from the donor or subject 102, where the donor or subject 102 may be stuck with a lumen needle or other device, allowing whole blood to flow from the donor or subject 102 into the apheresis system 200 and allowing blood components or constituents to flow back to the donor or subject 102. The tubing 108A proceeds to the soft cassette 340. In at least one example embodiment, a donor air detection sensor (ADS) 312 can be placed on or in the tubing 108A. The donor air detection sensor may be configured to detect the presence of fluid and/or air within tubing 108A.

The soft cassette 340 may include a first cassette port 360A where the tube 108A breaks into first and second tubing sections. For example, the first cassette port 360A can function as, include, and/or be substantially proximate to a “Y” connector or section that separates the tubing 108A into the first bypass branch 358A and the first tubing section 368A. The two tubing sections and can reconnect at the second cassette port 360B, which like the first cassette port 360A, can function as, include, and/or be substantially proximate to a “Y” connector or section. In at least one example embodiment, the first tubing may be bisected by a fluid sensor 316 forming a first bypass branch 358A and a second bypass branch 358B. In at least one example embodiment, the first bypass branch 358A may include a first fluid control valve 320C. In at least one example embodiment, the second tubing may be bisected by the drip chamber 354 forming a first tubing section 368A and a second tubing section 368B. The drip chamber 354 may be configured to collect a volume of whole blood and/or high hematocrit blood (blood with a high percentage of red blood cells) depending on the operation of the system 200. In at least one example embodiment, the first tubing section 368A may include a second fluid control valve 320A, while the second tubing section 368B includes a third fluid control valve 320B. The fluid control valves 320A, 320B, 320C may be configured to isolate the different tubing portions 358A, 368A, 358B, 368B.

The inlet tubing 108B may be connected to the second cassette port 360B and may connect the soft cassette 340 to the flexible loop 524. In at least one example embodiment, the inlet tubing 108B may include a sensor 808, disposed on or in the tubing 108B before a system static loop connector 528 of the flexible loop 524. In at least one example embodiment, the inlet tubing 108B may include a pressure sensor 808 disposed between the second cassette port 360B and the system static loop connector 528. In each instance, a draw pump 208 may be configured to pumped fluid through the tubing 108B either away from the soft cassette 340 or towards the soft cassette 340.

Two or more different tubes may be connected to the flexible loop 524 via the system static loop connector 528 and may provide fluid to, or receive fluid from, a blood component collection bladder 536. In at least one example embodiment, an exit tubing 112 may be connected to the flexible loop 524 via the system static loop connector 528. In at least one example embodiment, the exit tubing 112 may include another line sensor 812 that is disposed thereon or therein and is configured to detect fluid, air, cellular concentration, color, and/or color change in the fluid moving from the flexible loop 524. In at least one example embodiment, a second pressure sensor or fluid sensor 816 may also be disposed in or on line 112. The sensor 816 may be configured to detect one or more of the presence or absence of fluid within the exit tubing 113, pressure within the exit tubing 112, and/or other characteristics of the fluid in exit tubing 112. In each instance, the exit tubing 112 may flow into a plasma air detection sensor 284 before a saline and plasma tubing y-connector 280 separates the exit tubing 112 into saline tubing 116 and plasma tubing 120. In at least one example embodiment, a return pump 212 may engage with the exit tubing 112 to cause fluid or air to flow through the exit tubing 112 from either the flexible loop 524 or a saline bag 118 and/or a collection container 122.

FIG. 5 illustrates an example electrical and control system 900 configured to control the functions of the apheresis system 200. Generally, the control system 900 may include one or more nodes, which can include various hardware, firmware, and/or software configured to control and/or communicate with the mechanical, electromechanical, and electrical components of the apheresis system 200. Each node may be configured to control a different part of the apheresis system 200. For example, the control system 900 may include a cassette node 904 which may be a soft cassette assembly system may be configured to control and/or communicate with the components of a fluid component collection set (and/or the associated hardware or mechanical components interfacing with fluid component collection set or components thereof). The centrifuge node 908 which may be a centrifuge system may be configured to control and/or communicate with the centrifuge assembly 400 (and/or the associated hardware or mechanical components associated the with the centrifuge assembly 400 or components thereof). In at least one example embodiment, the cassette node 904 and centrifuge node 908 may be in communication with each other either wirelessly or through some other electrical or data connection. In at least one example embodiment, the cassette node 904 and centrifuge node 908 may separate portions of a single node or system 902. In at least one example embodiment, the cassette node 904 and centrifuge node 908 may have the same or different physical hardware that is used to operate and/or control different functions.

Each of the cassette node 904 and the centrifuge node 908 may be in communication with one or more sensors 916, 920, and/or 924. There may be more or fewer sensors than those shown in FIG. 5, as represented by ellipsis 928. Each of the cassette node 904 and/or the centrifuge node 908 can communicate directly to each sensor 916-924, or in other embodiments, may communicate with the several sensors 916-924 via a bus 912. The bus 912 may communicate by any type of communication protocol, such as universal serial bus (USB), a universal asynchronous receive/transmit (UART), or other types of bus systems or parallel communication connections. The sensors 916-924 can be any type of sensor that can communicate information, for example, about light, fluid, the presence of air, color, and/or pressure. Some of the sensors 916-924 may include sensors such as the air detection sensor 312, the fluid sensor 316, the anticoagulant air detection sensor 804, the pressure sensor 808, the line sensor 812, the second pressure sensor 816, and/or the air detection sensor 284.

Each of the cassette node 904 and the centrifuge node 908 may be in communication with one or more pump drives, pump motors, 936, 940, 944 (referred to collectively as “pumps”). There may be more or fewer pumps than are shown in FIG. 5, as represented by ellipsis 948. The cassette node 904 and/or the centrifuge node 908 may communicate with the pumps 936-944 through direct wired or wireless communication or through a bus 932. The bus 932 can be a control area network (CAN) bus, universal serial bus, or other type of bus architecture to communicate with the pumps 936-944. The pumps 936-944 can include, or be a part of, at least one of the draw pump 208, the return pump 212, and the anticoagulant pump 216.

FIG. 6 illustrates an example cassette node 904. As illustrated, in at least one example embodiment, the cassette node 904 may include one or more of a controller 1004, a memory 1008, a valve controller 1020, a communication interface for a control area network bus 1016, a communication interface for a universal asynchronous receive/transmit 1012, and a communication interface for other types of buses. Although not illustrated, it should be recognized that the cassette node 904 can also include other hardware, firmware, and/or software.

The controller 1004, which may also be referred to as a processor, may be any type of microcontroller, microprocessor, Field Programmable Gate Array (FPGA), Application Specific Integrated Circuit (A SIC), etc. An example controller 1004 may be the NK 10DN 512V OK 10 microcontroller, made and sold by N9P USA, Incorporated, which is a microcontroller unit with a 32-bit architecture. Other types of controllers, however, are possible. In each instance, the controller 1004 is configured to control other types of devices and/or to direct the functions of other types of devices, such as valves such as the first fluid control valve 320A, the second fluid control valve 320B, the draw fluid control valve 320C, the plasma flow control valve 286, the saline flow control valve 288, and/or the pumps 936-944. The controller 1004 may also be configured to communicate with various sensors 916-924 or other devices to receive and/or send information regarding the function of the apheresis system 200.

Other examples of the controller 1004, may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and 17-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, ARM® Cortex™-M processors, ARM® Cortex-A and ARM 926EJ-S™ processors, other industry-equivalent processors, and may perform computational functions using any known or future-developed standard, instruction set, libraries, and/or architecture.

The memory 1008 can be any type of memory including random access memory (RAM), read only memory (ROM), electrically erasable programmable ROM (EEPROM), a portable compact disc read-only memory (CD-ROM), an optical storage device, a magnetic storage device, any suitable combination of the foregoing, or other type of storage or memory device that stores and provides instructions to program and control the controller 1004. The memory 1008 may provide all types of software or firmware that programs the functions of the controller 1004.

The controller 1004 can communicate with one or more valve controllers 1020. Valves, such as such as the first fluid control valve 320A, the second fluid control valve 320B, the draw fluid control valve 320C, the plasma flow control valve 286, and/or the saline flow control valve 288, may be controlled by a valve controller 1020 and may be associated with a component of the system 200. The valve controller 1020 can provide the electrical signal, operational directive, or power to close or open any one of the valves, for example, the saline and plasma valve housing 276, the plasma flow control valve 286, the saline flow control valve 288, the first fluid control valve 320A, the second fluid control valve 320B, and/or the draw fluid control valve 320C.

The controller 1004 can also be connected to a bus 912, 932 through transceivers 1012, 1016 provided outside of the controller 1004 or integral to the controller 1004. The universal asynchronous receive/transmit transceiver 1012 may communicate with one or more of the sensors 916-924 or other devices. Likewise, the control area network bus transceiver 1016 can communicate with one or more of the pump controllers 936-944 or other devices. universal asynchronous receive/transmit transceivers 1012 and busses and control area network bus transceivers 1016 and busses are well known in the art and need not be explained further herein.

FIG. 7 illustrates an example centrifuge node 908. The centrifuge node can include the same or similar types of components as the cassette node 904. For example, in at least one example embodiment, as illustrated, the centrifuge node 908 may include a controller 1104 and/or a universal asynchronous receive/transmit transceiver 1112. Similar to the controller 1004, in at least one example embodiment, the controller 1104 may include any type of processor or microcontroller, including, for example, the N K 10D N 512V OK 10 microcontroller unit with 32-bit architecture from N9P USA, Incorporated.

In at least one example embodiment, the controller 1104 may be configured to communicate with the sensors 916-924, for example, directly, through the universal asynchronous receive/transmit transceiver 1112 and/or through other busses or systems. In at least one example embodiment, the controller 1104 may be configured to communicate with a brake controller 1124. The brake controller 1124 may be configured to brake or slow and stop the centrifuge 400. In at least one example embodiment, the controller 1104 may be configured to communicate with a motor transceiver 1116. The motor transceiver 1116 may be configured to communicate with a motor power system or a motor controller that functions to spin up or rotate the centrifuge 400 and/or control the speed setting or other function of the centrifuge 400. In at least one example embodiment, the controller 1104 may be configured to communicate with a cuff controller 1120 that can change or set the pressure of a pressure cuff on a donor or subject's 102 arm during the apheresis process.

In at least one example embodiment, the controller 1104 may be configured to communicate a strobe light 1114 which can be any light that flashes at a periodicity in synchronicity with the rate of spin of the motor, such that an operator of the apheresis system 200 can see the operation of the filler 460. The controller 1104 may be configured to communicate with the strobe light 1114 so as to change the frequency of the flashing of the strobe light 1114 and/or the intensity of the strobe light 1114.

FIG. 8 is a flowchart illustrating an example data entry process 1200, for example, for initializing the apheresis system 200. The data entry process 1200 may help to ensure, for example, that a target amount or volume of plasma and/or other blood component or constituents based on donor information is obtained and/or the quality of the plasma and/or other blood component or constituents. Also, in at least one example embodiment, that data entry process 1200, may allow for entry of other information, like bottle identification information and/or fluid component collection set such that the apheresis system 200 is capable of recording an indication as to which bottle and/or fluid component collection set was used for which donor or subject 102. In at least one example embodiment, the data entry process 1200 may begin at 1202 where the apheresis system 200 is powered on to await a new donor or subject 102.

In at least one example embodiment, as illustrated in FIG. 9, the apheresis system 200 may include an integrated identification reader (eg., radio-frequency identification (RFID) reader, barcode reader, etc.) 1221 that is configured to read a code (eg., radio-frequency identification tag, barcode, etc.) associated with a particular donor or subject 102 and/or a code (eg., radio-frequency identification tag, barcode, etc.) associated with a with a one or more components of a fluid component collection set and/or a code (eg., radio-frequency identification tag, barcode, etc.) associated with a collection container to control and/or determine certain operation parameters of the apheresis system 200 in accordance with the information received by the identification reader. The information and/or data may include donor-specific information (eg., biological information, including, for example, age, weight, height, hematocrit, hemoglobin, and/or donor history) and/or other information which may be relevant to the donation process (eg., identity and/or relationship of one or more or more components of a fluid component collection set and/or collection container.

In at least one example embodiment, the apheresis system 200 may include one or more computer systems. For example, as illustrated in FIG. 10, the apheresis system 200 may include one or more computer systems 1627. The computer system(s) 1627 may including, for example, a processor 1630, memory 1633, input/output devices 1636, one or more pump control systems 1639, one or more sensors 1642, and/or other appropriate elements. The processor 1630 may be configured to execute software. The software may include firmware, applications, and/or operating systems which may manage execution of the apheresis system 200. The computer system(s) 1627 may be configured to detect the start 1202 of the apheresis system 200 and/or may be configured to transmit certain information and/or data to a server 1621 via a connection to a network 1618, as illustrated in FIG. 11. In this manner, the apheresis system 200 may communicate certain information and/or data (eg., a data log, a firmware version identifier, and an error log) with a local system, such as a computer on location at a donation site, which may also be configured to communicate with the server 1621.

With renewed reference to FIG. 8, in at least one example embodiment, the data entry process 1200 may include receiving 1203 the donor-specific information and/or data and/or receiving 1209 information and/or data associated with the one or more components of a fluid component collection set and/or receiving 1212 information and/or data associated with the collection container. For example, the apheresis system 200 may be configured to scan a barcode, quick release code, or other type of image to receive donor-specific information, for example, from an identification card, and/or to received specific information about the one or more components of a fluid component collection set and/or collection container.

In at least one example embodiment, after receiving 1203 the donor-specific information and/or receiving 1209 the data associated with the one or more components of a fluid component collection set and/or receiving 1212 the data associated with the collection container, the apheresis system 200 may be configured to confirm receipt of the information and/data using a feedback system, such as a graphical user interface (GUI) 1230, as illustrated in FIG. 12. In this manner, a nurse, practitioner, or other user or operator of the apheresis system may be able to quickly ascertain whether the donor information and/or data has been properly inputted into the apheresis system 200. In at least one example embodiment, the feedback system may also, or alternatively, include a speaker that may be configured to provide audible feedback.

With renewed reference to FIG. 8, in at least one example embodiment, the method 1200 may further include, using the received donor-specific information and/or data to identify 1206 the donor or subject 102. For example, in at least one example embodiment, the apheresis system 200 (for example, using the control system 900 and/or the computer system(s) 1627) may be configured to identify 1206, using the information and/or data received via the scanner 1221, whether the donor or subject 102 is associated with any existing donor or subject 102 identity in a database and/or whether the donor or subject 102 is a new donor or subject.

The method 1200 may also include receiving 1209 information and/or data associated with a fluid component collection set to be used during the donation process. In at least one example embodiment, the fluid component collection set may include a soft cassette assembly, like the soft cassette assembly 300. In at least one example embodiment, the information and/or data associated with the fluid component collection set may be received 1209 by the apheresis system 200 via a barcode, quick release code, radio-frequency identification chip, and/or other type of scannable object that may be associated with the fluid component collection set. In at least one example embodiment, each fluid component collection set may be affixed with a label or sticker that include a distinct barcode, quick release code, radio-frequency identification chip, and/or other type of scannable object. By scanning the label or sticker on the fluid component collection set, the apheresis system 200 may be enabled to record into memory (and in at least one embodiment, shared with the server 1621) which fluid component collection set is being used for the current donation process. In this manner, the apheresis system 200 may be enabled to associate donor with a selected fluid component collection set and/or one or more components thereof. The data associated with the fluid component collection set may include, for example, a date of manufacture, an identity of manufacturer, and/or various other information that may be useful for data processing purposes after the donation is complete. In at least one example embodiment, data associated with the fluid component collection set received 1209 through scanning may be used to determine a type of fluid component collection set. The type of fluid component collection set may be used by the apheresis system 200 to adjust one or more settings such as flow rate or other information during the donation process.

In at least one example embodiment, after scanning the fluid component collection set, an operator of the apheresis system 200 may receive confirmation of the receipt of the information from the fluid component collection set. For example, a graphical user interface 1230, as illustrated in FIG. 12, may display an indication as to whether the information and/or data from a fluid component collection set has been received. In at least one example embodiment, instead of or in addition to displaying through a graphical user interface 1230, the apheresis system 200 may play an audible sound through one or more speakers or display lights of various colors to indicate the data has been received.

With renewed reference to FIG. 8, in at least one example embodiment, the method 1200 includes receiving 1212 data associated with a collection container. For example, in at least one example embodiment, to initialize the apheresis system 200 for a new donor, a collection container may be required. After donation, the collection container may be filled with the donated plasma (or other fluid). For data tracking purposes, the collection container may be required to be associated with the donor making it may be necessary for an identity of the collection container to be recorded. As such, an operator of the apheresis system 200 may be enabled to scan a label, sticker, or other object associated with (eg., printed on or sticked to) the collection container using the apheresis system 200. As with the previously detailed steps, upon receiving 1212 the information and/or data from a plasma collection container, the apheresis system 200 may acknowledge receipt of the data via a graphical user interface 1230, speaker, white, and/or other feedback system.

In at least one example embodiment, the method 1200 may use the received data and/or information (including the donor-specific information and/or data and/or the blood component collect set information and/or data and/or the collection container information and/or data) to determine and/or selected 1213 one or more operating parameters of the donation process and then to optionally continue or end the collection process at 1215. For example, in at least one example embodiment, the apheresis system 200 may be configured (for example, via the control system 900 and/or the computer system(s) 1627) to calculate the amount or volume of plasma (or other fluid) the donor or subject 102 can provide or donate using the information and/or data received, such as further detailed in Atty. Docket No. 18955-000224-US-PS1, titled METHODS AND SYSTEMS FOR DETERMINING DONOR SPECIFIC AMOUNTS FOR COLLECTION USING TARGETED CHANGES IN HEMATOCRIT and listing John Pittinger and Thomas J. Felt as inventors, as filed Apr. 29, 2023 and assigned U.S. App. No. 63/462,636, the entire contents of which are herein incorporated by reference.

In at least one example embodiment, the method 1200 may end at 1218, at which point the donation process may continue with the extraction of fluids from the donor being completed. Any information and/or data received through the above discussed steps may be recorded into memory and/or shared with one or more computer systems 1627. For example, a database entry may be created for the particular donation, including information such as an amount or volume of plasma extracted from the donor, a current weight of the donor, a time and/or date of the donation, and/or other information and/or data.

In various aspects, after the collection of the one or more blood components or constituents (e.g., plasma) using the apheresis system 200, it may be desirable to remove one or more samples from the collection. The one or more samples may be used to confirm the quality of the collection. The present disclosure provides inline sampling methods and devices for various apheresis systems (like the apheresis system 200). Sampling of the collection may help to ensure sample consistency and quality, to ensure and enforce safe, accepted collection levels, and monitoring collection processes. Sampling may occur after a collection process is complete. In at least one example embodiment, as discussed above, the apheresis system 200 includes a graphical user interface (GUI) (eg., graphical user interface 1230). The graphical user interface may be used to prompt the nurse, practitioner, or other user or operator of the apheresis system (eg., apheresis system 200) to complete the inline, post-collection sampling prior to removing the collection container (eg., collection container 122) and/or the tubing set from fluid communication with the apheresis system. In at least one example embodiment, the apheresis system (e.g., apheresis system 200) may be configured to prevent the removal of the collection container (eg., collection container 122) and/or the tubing set (or a component thereof) from fluid communication with the apheresis system if the sampling has not occurred or if a sample volume is less than a predetermined or desired sample amount. In at least one example embodiment, the collection container (e.g., collection container 122) may be removed from fluid communication with the apheresis system before the removal of the tubing set (or a component thereof) from fluid communication with the apheresis system.

FIG. 13 is a flowchart illustrating an example inline sampling method 1300 for an apheresis system, like the apheresis system 200. The method 1300 includes collecting (or taking) 1340 a sample from the collection (i.e., the one or more isolated blood components or constituents). In at least one example embodiment, the collection 1340 may include establishing 1344 fluid communication between a fluid component collection set including the collection container and a sample tube. Establishing 1344 fluid communication between the fluid component collection set and the sample tube may include placing the sample tube in line with the fluid component collection set or joining the sample tube to the existing fluid component collection set. In at least one example embodiment, the sample tube may be part of an originally placed fluid component collection set. In other example embodiments, fluid communicating may be established 1344 between the fluid component collect set and the sample tube. In each instance, the collection 1340 may include removing 1348 the sample tube from fluid communication with the fluid component collection set.

In at least one example embodiment, the collection 1340 may further include verifying 1346 that an appropriate sample volume has been collected. The appropriate sample volume may be a predetermined or desired sample amount. In at least one example embodiment, verifying 1346 the appropriate sample volume may include taking a weight of the sample tube including the collected or isolated sample, for example using a scale provided as part of the apheresis system. If the weight is as expected, or as determined, the sample volume may be verified. If the weight is not as expected, or as determined, the sample volume may not be verified. In at least one example embodiment, verifying 1346 that the appropriate sample volume is collected may be an ongoing process from the time that fluid communication has been established to the time that the sample tube is removed. Although not illustrated, it should be appreciated that, in various example embodiments, the sample method 1300 may include alerting, for example using a graphical user interface, the nurse, practitioner, or other user or operator of the apheresis system that the appropriate sample amount has been collected, and additionally, or alternatively, alerting the nurse, practitioner, or other user or operator of the apheresis system that appropriate sample amount has not yet been collected and instruction the nurse, practitioner, or other user or operator of the apheresis system, for example, to place in-line another sample tube.

In at least one example embodiment, the collection 1340 may further include prompting 1342 the nurse, practitioner, or other user or operator of the apheresis system to establish 1344 the fluid communication between the fluid component collection set and the sample tube and/or to remove 1348 the sample tube from fluid communication with the fluid component collection set. The nurse, practitioner, or other user or operator of the apheresis system may be prompted 1342 using a graphical user interface of the apheresis system of the apheresis system. After the sample is collected 1340, the method 1300 may further include unloading the collection container and the separation set from the apheresis system and ending the procedure.

In at least one example embodiment, the sampling method 1300 may include, before the sample collection, removing 1320 needles (or other connectors) from the donor (or subject). The sampling method 1300 may also include sealing the lines and confirming 1322 completion of the same. The nurse, practitioner, or other user or operator of the apheresis system may confirm 1322 removal and sealing using a graphical user interface of the apheresis system, for example, by selecting a confirmation button and/or completing a checklist. Further still, the method 1300 may include, before the removing 1320 needles (or other connectors) from the donor and/or sealing the lines and the confirming 1322 completion of the same, confirming 1310 that collection is complete. The confirming 1310 may require the nurse, practitioner, or other user or operator of the apheresis system to interact with the graphical user interface, for example, by selecting a confirmation button and/or completing a checklist. Confirming the completion of the collection, removing needles from the donor and sealing lines, and confirming needle removal and line sealing may help to ensure the safety of the donor or subject. In at least one example embodiment, the apheresis system may use a pressure sensor to confirm disconnection and sealing of the various lines.

In each instance, after the sample is collected 1340, the method 1300 may further include unloading the collection container and the fluid component collection set from the apheresis system and ending the procedure.

FIG. 14 is an illustration of an example fluid component collection set 500 that can be used with an apheresis system, like the apheresis system illustrated in FIG. 1, and for inline sampling methods, like method illustrated in FIG. 13. As illustrated, the fluid component collection set 500 includes various connections that include, for example, tubes and connectors. For example, as illustrated, the blood component collection set 500 may include one or more tubes, such as the cassette inlet tubing 108A, the loop inlet tubing 108B, the anticoagulant tubing 110, the loop exit tubing 112, the saline tubing 116, and/or the plasma tubing 120, and also one or more connectors, such as the tubing connector 106 and/or the saline and plasma tubing y-connector 280. The fluid component collection set 500 may also include one or more other connectors, such as a first tubing fitting 504, a second tubing fitting 508, a bag fitting 512, a system static loop connector 528, and/or a filler loop connector 532. The various connections may fluidly connect the soft cassette 340 and the blood component collection loop 520.

The one or more tubes, including the cassette inlet tubing 108A, the loop inlet tubing 108B, the anticoagulant tubing 110, the loop exit tubing 112, the saline tubing 116, and/or the plasma tubing 120 (collectively referred to as “the tubing”), each have a central lumen configured to convey fluid therethrough. The tubing may include one or more polymeric materials, including, for example, polyvinyl chloride (PVC), plasticized-polyvinyl chloride, polyethylene, ethylene vinyl acetate (EVA), rubbers, copolymers and combinations thereof.

The one or more connectors, including the tubing connector 106, the saline and plasma tubing y-connector 280, the first tubing fittings 504, the second tubing fitting 508, the bag fitting 512, the system static loop connector 528, and/or the filler loop connector 532 (collectively referred to as “the connectors”), may be each configured to fluidly interconnect the tubing and/or to fluidly interconnect the tubing and other medical accessories and/or to fluidly interconnect the tubing and needles or spikes. For example, the connectors may insert into the central lumen of the respective tube and/or attach to an outside of the respective tube and/or the bag fitting 512 may be configured to be inserted into a receiving bag, like the saline bag 118. In at least one example embodiment, the connectors may include various fittings, including, for example, Luer fittings, twist-to-connect fittings, and/or other small-bore couplings, to provide universal and/or reliable interconnections for establishing fluid connections.

In at least one example embodiment, the blood component collection loop 520 may include a flexible loop 524 disposed between the system static loop connector 528 and the filler loop connector 532. The static loop connector 528 may be attached to the flexible loop 524 and/or a blood component collection bladder 536, as further discussed below, by a mechanical lock, which can be formed with a photo curable adhesive. The flexible loop 524 may be configured as a hollow flexible tube configured to receive and/or contain at least a portion of the loop inlet tubing 108B and the loop exit tubing 112. In at least one example embodiment, the flexible loop 524 may include a thermoplastic elastomer having enhanced flexibility for transmitting twist from a first end of the flexible loop 524 towards and to a second distal end. Such thermoplastic elastomers may provide the flexibility of rubber while maintaining the strength and torque characteristics of plastics. Examples of the thermoplastic elastomer may include, for example, copolyester, DUPONT™ HYTREI® thermoplastic elastomers, EASTMAN NEOSTAR™ elastomers, CELANESE RITEFLEX® elastomers, TOYOBO PELPRENE®, and/or other similar brand elastomers offering high flexibility and strength characteristics.

In at least one example embodiment, the blood component collection loop 520 may include a blood component collection bladder 536. The blood component collection bladder 536 may have a first or bladder loop end 540A and a second or bladder free end 540B. The blood component collection bladder 536 may include a first collection flow chamber 544 extending between the bladder loop end 540A and the bladder free end 540B and connected to the flexible loop 524 via the filler loop connector 532. For example, in at least one example embodiment, fluid may flow between the loop inlet tubing 108B and the first collection flow chamber 544 via the flowpath defined by the flexible loop 524, the system static loop connector 528, and the filler loop connector 532. The bladder free end 540B of the first collection flow chamber 544 may include a flow chamber transition 548 and fluid flowing from the bladder loop end 540A to the bladder free end 540B via first collection flow chamber 544 may enter a second collection flow chamber 552 via the flow chamber transition 548. The second collection flow chamber 552 may be connected to the flexible loop 524 via the filler loop connector 532. For example, in at least one example embodiment, fluid may flow between the loop exit tubing 112 and the second collection flow chamber 552 via a flowpath defined by the flexible loop 524, the system static loop connector 528, and the filler loop connector 532.

A sample tube (such as the sample tube 1530 illustrated in FIGS. 15A-15C) may be connected to the fluid component collection set 500 in place of, or in addition to, the first tubing fitting 504. For example, in at least one example embodiment, the sample tube may be couplable to the tube fitting 504, while in another example, the sample tube may be couplable to the plasma tubing 120 before the collection container (e.g., collection container 122), for example, in place of, or in addition to, the tube fitting 504. In at least one example embodiment, the sample tube may be couplable to the fluid component collection set 500 via a tee connector. In at least one example embodiment, the fluid component collection set 500 may be couplable to a receiving sleeve and the sample tube may be couplable to the receiving sleeve. In at least one example embodiment, the fluid component collection set 500 may include the receiving sleeve that receives the sample tube. As mentioned above, in at least one example embodiment, the sample tube may be provided with the fluid component collection set 500. In at least one example embodiment, the sample tube may be separate from the fluid component collection set 500.

FIGS. 15A-15C are illustrations of an example sampling system 1500 that can be used for inline sampling methods, like the method illustrated in FIG. 13, in accordance with at least one example embodiment of the present disclosure. In at least one example embodiment, the sampling system 1500 may be a vacuum tube assembly that includes a sample sleeve 1510 and a sample tube 1530 that may be at least partially received by the sample sleeve 1510. The sample sleeve 1510 may include an outer housing 1512 having a first end 1514 and an opposing second end 1516, where the first end 1514 is configured to be joined to a fluid component collection set (like the fluid component collection set 500), and more specifically, a plasma tubing line (like the plasma tubing 120) of the fluid component collection set and the second end 1516 is configure to at least partially receive the sample tube 1530. In at least one example embodiment, as illustrated, the first end 1514 of the sample sleeve 1510 may be couplable to the fluid component collection set via a tee connector 1550. The tee connector 1550 may be provided with the fluid component collection set. For example, in at least one example embodiment, the tee connector 1150 and/or the sample sleeve 1510 may be formed integrally with the fluid component collection set. In other example embodiments, the tee connector 1150 and/or the sample sleeve 1510 may be coupled to the fluid component collection set when fluid communication is initiated between the fluid component collection set and the collection system (eg., apheresis system). In other example embodiments, the tee connector 1150 and/or the sample sleeve 1510 may be coupled to the fluid component collection set when after fluid communication is initiated between the fluid component collection set and the collection system (eg., apheresis system).

In at least one example embodiment, the first end 1514 of the sample sleeve 1510 may include a cap 1518 and a conduit 1520 extending therethrough. The conduit 1520 may include a first end 1522 and an opposing second end 1524, where the first end 1522 is couplable to the fluid component collection set (like the fluid component collection set 500), and more specifically, the plasma tubing line (like the plasma tubing 120) of the fluid component collection set and the second end 1524 may engage with and extend into the sample tube 1530. In at least one example embodiment, the first end 1522 of the conduit 1520 may include a coupler 1526 that couples the sampling system 1500 to the fluid component collection set. In at least one example embodiment, the coupler 1526 may be couplable to the fluid component collection set via a tee connector 1550.

In at least one example embodiment, the sampling system 1500 may include a seal 1532 that helps to secure the sample tube 1530 within the sample sleeve 1510 but allows vertical movement of the sample tube 1530 within the sample sleeve 1510. For example, FIG. 15A illustrates a first assembled position where the sample tube 1530 is at least partially received within the sample sleeve 1510 but no fluid communication has been established between the sample tube 1530 and the fluid component collection set; FIG. 15B illustrates a second assembled position where the sample tube 1530 is at least partially received within the sample sleeve 1510 fluid communication has been established between the sample tube 1530 and the fluid component collection set; and FIG. 15C illustrates a disconnected position where the sample tube 1530 is removed from, or not yet inserted into the sample sleeve 1510. The nurse, practitioner, or other user or operator of the apheresis system may apply an upward vertical force or pressure to move the sample tube 1530 from the assembled position as illustrated in FIG. 15A to the sampling position as illustrated in FIG. 15B and a downward vertical force or pressure to move the sample tube 1530 out of the sleeve 1510 and to the disconnected state as illustrated in FIG. 15C.

In at least one example embodiment, the sampling system 1500 may be included with, or as part of, a fluid component collection set (like the fluid component collection set 500). The sampling system 1500 may be provided in the first assembled position as illustrated in FIG. 15A and the nurse, practitioner, or other user or operator of the apheresis system will be required to apply a force to the sample tube 1530 to establish fluid communication between the sample tube 1530 and the fluid component collection set at an appropriate time. The sampling system 1500 may be provided in a disconnected state such as illustrated in FIG. 15C as part of a kit including the fluid component collection set (like the fluid component collection set 500) where the nurse, practitioner, or other user or operator of the apheresis system will be required to connect the sample sleeve 1510 to the fluid component collection set and to then introduce the sample tube 1530 and establish fluid communication between the sample tube 1530 and the fluid component collection set at an appropriate time. In each instance, the nurse, practitioner, or other user or operator of the apheresis system may be instructed to establish the fluid communication using graphical user interface of the apheresis system.

In at least one example embodiment, the sampling tube 1530 and/or the sampling sleeve 1510 may include a code 1560 (eg., radio-frequency identification tag, barcode, etc.) that establishes, or that can be used to establish, a relationship between the sampling tube 1530 and/or sampling sleeve 1510 and the fluid component collection set and/or the apheresis system and/or the donor or subject.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An in-line sampling method for medical device, the method comprising:
establishing fluid communication between a fluid component collection set as received by the medical device and a sample system for sample collection, the medical device prohibiting removal of the fluid component collection set or a component thereof from fluid communication with the medical device until the sample collection using the sample system is complete.

2. The method of claim 1, wherein the sample collection includes transferring a sample volume to the sample system and the sample collection is complete only after entire sample volume is transferred.

3. The method of claim 2, wherein the method further includes:
determining the sample volume based on a specific nature of a collection process using the medical device.

4. The method of claim 1, wherein the medical device includes a scale and the sample collection is complete when the sample system is found to have a selected weight.

5. The method of claim 1, wherein the method further includes:
prompting an operator to establish the fluid communication between the fluid component collection set and the sample system.

6. The method of claim 5, wherein the method further includes:
alerting the operator when sample collection is complete.

7. The method of claim 6, wherein concurrent with or after the alerting of the operator that the sample collection is complete, the method further includes:
prompting the operator to remove the sample system from fluid communication with the medical device.

8. The method of claim 7, wherein the prompt to the operator to remove the sample system from fluid communication with the medical device includes prompting the operator to remove the sample system from fluid communication with the fluid component collection set.

9. The method of claim 7, wherein concurrent with or after the prompting of the operator to remove the sample system from fluid communication with the medical device, the method further includes:
prompting the operator to remove a collection container from fluid communication with the medical device and from fluid communication with the fluid component collection set.

10. The method of claim 9, wherein the medical device prohibits removal of the collection container from the medical device and from fluid communication with the fluid component collection set until the sample collection using the sample system is complete.

11. The method of claim 9, wherein after the removal of the collection container from the medical device, the method further comprises:
prompting the operator to remove the fluid component collection set from fluid communication with the medical device.

12. The method of claim 1, wherein before the establish of the fluid communication between the fluid component collection set as received by the medical device and the sample system for sample collection, the method further includes:
prompting an operator to confirm removal of one or more connectors from a subject, the one or more connectors fluidly connecting the medical device and the subject for the sample collection.

13. The method of claim 1, wherein the sampling system includes:
a sample sleeve having a first end and an opposing second end, the first end being couplable to the fluid component collection set;
a sample tube configured to be at least partially received by the second end of the sample sleeve; and
a sealing member for securing the sample tube within the sample sleeve while allowing for movement of the sample tube from a connected state where there is fluid communication between the sample tube and the fluid component collection set and a disconnected state where there is no fluid communication between the sample tube and the fluid component collection set.

14. The method of claim 13, wherein the first end of the sample sleeve is couplable to the fluid component collection set using a tee connector as provided with the fluid component collection set.

15. The method of claim 13, wherein the first end of the sample sleeve includes a cap and a conduit that extends therethrough, the conduit including a first end and an opposing second end, the first end of the conduit being configured to couple the sample sleeve to the fluid component collection set and the second end of the conduit configured to engage with and extend into the sample tube.

16. The method of claim 13, wherein the method further includes:
prompting the medical device to interpret a code disposed on an exterior-facing surface of the sample tube.

17. A method for in-line sampling for a medical device, the method comprising:
prompting an operator of a medical device to establish a fluid communication between a fluid component collection set as received by the medical device and a sample tube for sample collection, the medical device prohibiting removal of the fluid component collection set or a component thereof from fluid communication with the medical device until sample collection using the sample tube is complete and the establishing of fluid communication between the fluid component collection set includes prompting the medical device to interpret a code disposed on an exterior-facing surface of the sample tube.

18. The method of claim 17, wherein the fluid component collection set includes:
a sample sleeve configured to receive the sample tube; and
a sealing member for securing the sample tube within the sample sleeve while allowing for movement of the sample tube from a connected state where there is fluid communication between the sample tube and the fluid component collection set and a disconnected state where there is no fluid communication between the sample tube and the fluid component collection set.

19. The method of claim 17, wherein before the prompting of the operator to establish fluid communication between the fluid component collection set as received by the medical device and the sample tube for sample collection, the method further includes:
prompting the operator to confirm removal of one or more connectors from a subject, the one or more connectors fluidly connecting the medical device and the subject for the sample collection.

20. The method of claim 17, wherein the method further includes:
alerting the operator when sample collection is complete, the medical device including a scale and the sample collection is complete when the sample tube is found to have a selected weight; and
prompting the operator to remove the sample tube from fluid communication with the medical device.
